(19) United States
(12) Patent Application Publication
(10) Pub. No.: US 2004/0098756 A1
(43) Pub. Date: May 20, 2004

(54) Transgenic animals for use in protein expression and antibody production

(76) Inventor: Sujay Singh, San Diego, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/696,162

(22) Filed: Oct. 29, 2003

Related U.S. Application Data
(60) Provisional application No. 60/422,056, filed on Oct. 29, 2002.

(51) Int. Cl. A01K 67/027; C12N 15/86

(52) U.S. Cl. 800/14; 800/15; 800/16; 800/17; 800/18; 800/19; 800/20; 435/456

(57) ABSTRACT

The present invention concerns transgenic vertebrates that are useful in expressing proteins and in producing antibodies. The present invention discloses methods for producing vertebrates that are transgenic for a bacteriophage RNA polymerase. The present invention further discloses methods for the use of such transgenic vertebrates in protein expression and in antibody production.

The present application claims priority from U.S. Provisional Patent Application No. 60/422,056 to Singh, entitled “Transgenic animals for use in antibody production”, filed on 29 Oct. 2002, which is incorporated by reference herein in its entirety.

This invention was made with government support awarded by the National Institutes of Health, National Institute of General Medical Sciences, SBIR Grant No. 1R43GM64253-1A1. The United States Government may have certain rights in the invention.

### TECHNICAL FIELD

The present invention relates generally to the field of genetic engineering and more specifically concerns methods for the production and use of transgenic vertebrates.

### BACKGROUND

The development of innovative, accessible, and easy-to-use research reagents designed for the study of protein function and structure is crucial in realizing the full utility of completed genome projects. Study of the complete protein set (proteome) encoded by a genome has advanced beyond the early definitions of proteomics or functional genomics, and is now embraced by many academic and corporate research laboratories world-wide. The pattern of protein expression changes during development and disease states, and depends on the organism's physiological state. Linking expression of the proteome to physiological changes associated with healthy or disease conditions is believed to be a way to identify clinically relevant molecular disease targets and developing novel drugs against them.

Antibodies are important tools to study the structure and function of proteins. Monoclonal antibodies are traditionally developed by immunizing mice with recombinant proteins or synthetic peptides conjugated to a protein carrier such as keyhole limpet hemocyanin (KLH) or bovine serum albumin (BSA), or with recombinant fusion proteins (for example, a protein antigen fused to glutathione S-transferase). The splenocytes from the immunized mice are fused with myeloma cells to obtain immortalized hybridoma clones. The immunization and hybridoma production process involves preparation of proteins, such as recombinant proteins, as antigens. Synthesis of peptides partially automates the production of antigens. However, the common practice of preparing antigens as recombinant proteins or synthetic peptides conjugated to carrier proteins is a time-consuming process, involving cloning of the target gene in a plasmid vector, introducing the plasmid into a bacterial or insect cell system, and purification of the recombinant protein using an affinity column. There may be a problem with solubility of the recombinant protein, and often, the immune response to the carrier protein dominates. Furthermore, since it is hard to predict the functional sites of many proteins, antibodies developed against synthetic peptides often are not useful in blocking or activating a protein function, which is one of the requirements of antibodies of therapeutic importance.

### BRIEF DESCRIPTION OF THE FIGURES

FIG. 1 depicts a non-limiting example of a construct including a bacteriophage RNA polymerase transgene that can be used in the present invention's methods to produce a transgenic vertebrate whose genome includes a bacteriophage RNA polymerase transgene. The schematic map depicts a vector that includes a eukaryotic expression plasmid (“pT7 RNAP”) containing a cytomegalovirus promoter (“CMV Promoter”), the bacteriophage T7 RNA polymerase gene (“T7 RNAP”) inserted into a polylinker sequence, and a sequence directing transcriptional termination derived from the SV40 virus poly-A signal sequence (“SV40 Poly-A”).

FIG. 2 depicts a non-limiting example of a system to obtain a construct for use in expressing a protein or in producing an antibody against an antigen in a transgenic vertebrate whose genome includes a bacteriophage RNA polymerase. Such constructs include a promoter sequence cognate to the bacteriophage RNA polymerase transgene, a eukaryotic ribosome recognition site, and a sequence encoding the protein or antigen to be expressed. The construct optionally includes any combination of a stop codon, a tag sequence, and a poly-adenosine tail. The construct may be linear or circular.

The specific system depicted yields a linear construct for use in expressing a protein or antigen in a vertebrate that is transgenic for T7 RNA polymerase. This construct includes, in order: a T7 RNA polymerase promoter (the cognate promoter sequence); a Kozak sequence (the eukaryotic ribosome recognition site); the sequence encoding the protein or antigen to be expressed; a sequence encoding a poly-histidine tag (the optional tag sequence); a stop codon; and a poly-adenosine tail. For each sequence encoding the protein or antigen to be expressed, a pair of oligonucleotide PCR primers is synthesized containing sequences specific to the protein or antigen to be expressed, and sequences required for proper expression. The 5′ primer contains (starting at the 5′ end) approximately 20 non-specific nucleotides (to provide a necessary structure for assembly of the transcriptional complex), a T7 promoter (also approximately 20 nucleotides), a Kozak sequence and 17-20 additional nucleotides corresponding to the beginning of the sequence encoding the protein or antigen to be expressed. The 3′ primer (starting at the 3′ end) contains 17-20 nucleotides corresponding to the end of the sequence encoding the protein or antigen to be expressed, 18 nucleotides (6 CAT repeats) corresponding to a hexa-histidine (“His6”) tag (to be used for purification of the protein and confirmation of protein production via western analysis), a stop codon for translation (TAA), and approximately 20 adenosines (“poly(A)”, to create a short poly-adenosine tail for added message stability).

### SUMMARY

The present invention provides methods to rapidly express and produce antibodies and other proteins through the use of vertebrates transgenic for a bacteriophage RNA polymerase. The bacteriophage RNA polymerase transgene allows transcription of RNA, such as mRNA transcribed from a PCR DNA product that includes the cognate RNA polymerase promoter. Any suitable bacteriophage RNA polymerase gene can be used, such as, but not limited to, T7 RNA polymerase, SP6 RNA polymerase, and T3 RNA polymerase. Much research has been done to study expression of T7 RNA polymerase (T7 RNAP) in different cell systems (Davanloo et al. (1984) Proc. Natl. Acad. Sci. USA, 81:2035-2039; Studier and Moffatt (1986) J. Mol. Biol., 189:113-130; and Studier et al. (1990) Methods Enzymol., 185:60-89, which are incorporated by reference in their entirety herein), and the properties of T7 RNAP are believed to make it suitable to generate antibodies against a large number of target antigens simultaneously.

Bacteriophage T7 is a virulent bacteriophage that infects E. coli. T7 RNA polymerase (T7 RNAP), the product of T7 gene 1, is a protein produced early in T7 infection (Tabor and Richardson (1985) Proc. Natl. Acad. Sci. USA, 82:1074-1078; and Tabor and Richardson (1992) Biotechnology, 24:280-284, which are incorporated by reference in their entirety herein). Bacteriophage T7 RNA polymerase is believed to be one of the simplest enzymes catalyzing RNA synthesis. In contrast to most known RNA polymerases, this enzyme consists of a single subunit and is able to carry out transcription in the absence of additional protein factors. The enzyme is widely used for synthesis of specific transcripts, and serves as a model for studying the mechanisms of transcription. T7 RNA polymerase interacts with its cognate promoter, a highly conserved sequence that is believed to consist of approximately 23 continuous base pairs (Rosa (1979) Cell, 16:815-825; and U.S. Pat. No. 4,952,496 to Studier et al., “Cloning and expression of the gene for bacteriophage T7 RNA polymerase”, issued 28 Aug. 1990, which are incorporated by reference in their entirety herein). T7 polymerase is very efficient at transcribing DNA containing the T7 promoter (Deng et al. (1991) Gene, 109:193-201).

A factor to consider in designing genetic constructs that contain a target gene or sequence for expression in eukaryotic cells is the translational efficiency of the target gene mRNAs. One feature of eukaryotic mRNAs is the presence of a 5′ cap consisting of a methylated guanylate residue. Also believed to be important for ribosome recognition is the eukaryotic signal sequence surrounding the AUG initiator codon, termed the Kozak sequence (see “Extracting Kozak Consensus Sequence Using Kleisli”, Chen et al., available on-line at www.bionet.nsc.ru/bgrs/thesis/61/, accessed 28 Oct. 2003; Kozak (1982) Biochem. Soc. Symp., 47:113-128; Kozak (1982) Jₓ Virol., 42:467-473, and Kozak (1987) Nucleic Acids Res., 15:8125-8148, which are incorporated by reference in their entirety herein). The ribosome complex-recognizes the first capped 5′ end of the mRNA, and scans for the Kozak sequence around the AUG eukaryotic initiation codon. Translation begins at the first AUG codon in the mRNA.

Genetic constructs that contain a target gene or sequence for expression in eukaryotic cells may be produced as linear constructs, such as polymerase chain reaction (PCR) products. For efficient translation of the PCR product, it is believed to be important to add the Kozak sequence to the PCR primer. Examination of the behavior of T7 RNA polymerase (RNAP) using a set of promoter variants having all possible single base-pair (bp) substitutions suggests that there is an absolute requirement for initiation with a purine and a strong preference for initiation with GTP versus ATP. Additional nucleotides can be added at the 5′-end of the sense primer, which contains a T7 promoter-binding site followed by a Kozak sequence. At the 3′-end the gene specific sequences are preceded by a terminator sequence.

T7 RNA polymerase is active in cells from different species, including mammalian, insect, fish, and amphibian cells. Some examples of this activity follow. T7 RNA polymerase has been shown to transcribe RNA from linear circular as well as circular DNA templates containing a T7 promoter. Microinjection of two components of the T7 RNA polymerase expression system into fertilized zebrafish eggs resulted in efficient expression of the reporter gene with levels of expression using the T7 RNA polymerase expression system similar to those obtained using a reporter gene under the control of a CMV promoter (Verri et al. (1997) Biochem. Biophys. Res. Commun., 237:492-495). Under the experimental conditions reported, survival of embryos was not affected and the embryos did not exhibit any obvious deformity, indicating that expression of T7 RNA polymerase did not interfere with normal embryonic development (Verri et al. (1997) Biochem. Biophys. Res. Commun., 237:492-495). A T7 RNA polymerase cancer gene therapy plasmid vector has been shown to inhibit tumor growth in mice (Chen et al. (1998) Hum. Gene Ther., 9:729-736). Others have demonstrated that a T7 RNA polymerase binary system can be used to express foreign genes in mammalian cells (Deng et al. (1991) Gene, 109:193-201; Blakely et al. (1991) Anal. Biochem., 194:302-308; Brisson et al. (1999) Gene Ther., 6:263-270; Chen et al. (1994) Nucleic Acids Res., 22:2114-2120). A hybrid recombinant baculovirus-bacteriophage T7 RNA polymerase expression system was developed for transient expression in insect cells of plasmids with foreign genes provided with a T7 promoter (van Poelwijk et al. (1995) Biotechnology (NY), 13:261-264; Kohl et al. (1999) Appl. Microbiol. Biotechnol., 53:51-56). The coding sequence for T7 RNA polymerase, with or without a nuclear localization signal, was inserted into the genome of Autographa californica nuclear polyhedrosis virus. Recombinant viruses stably expressed T7 RNA polymerase in insect cells. Upon transfection of infected insect cells with plasmids containing the genes for chloramphenicol acetyltransferase (CAT) and the hepatitis B virus precore-, core-, or e-antigens under control of the T7 promoter, transient expression of these genes was detected by ELISA (van Poelwijk et al. (1995) Biotechnology (NY), 13:261-264; Kohl et al. (1999) Appl. Microbiol. Biotechnol., 53:51-56).

T7 RNA polymerase is capable of beginning transcription outside the nucleus, thus avoiding the need for nuclear import of DNA. A T7 RNA polymerase expression system in insect or vertebrate (including mammalian, fish, or amphibian) cells thus contains everything necessary for gene expression, including materials similar to those found inside a cell's nucleus. Protein expression techniques, such as those used in gene therapy, can use only gene DNA; however, a T7 RNA polymerase method uses DNA prebound with T7 RNA polymerase, a protein that causes the gene to begin producing RNA, an important step in protein synthesis within a cell. Expression of a gene using a T7 RNA polymerase expression system is usually faster than expression obtained using a traditional plasmid DNA vector. Cytoplasmic (such as a T7 RNA polymerase expression system) and nuclear expression systems (such as those using plasmid vectors) utilize similar endocytosis pathways to the point of endosomal release. The cytoplasmic expression system shows immediate expression, once DNA is released into the cytoplasm, proportional to the amount of DNA released. In contrast, DNA targeted for nuclear expression requires additional time for nuclear entry. The level of nuclear expression is also restricted by the limited amount of DNA that is imported into the nucleus. Finally, mitosis is required for effective nuclear expression but not for cytoplasmic expression. Therefore, the cytoplasmic expression system, such as a T7 RNA polymerase expression system has considerable advantages over traditional nuclear expression systems and may be an effective method for transfecting nondividing cells.

Studies have shown that use of a linear DNA expression cassette results in long-term transgene expression in vivo with an expression level in mouse sera approximately 10- to 100-fold greater than expression in mice injected with closed circular DNA (Chen et al. (2001) Mol. Ther., 3:403-410). The expression level of protein from mice injected with linear PCR fragments containing a CMV promoter and a polyadenylation signal is comparable to mice immunized with circular plasmid, and the PCR fragments were observed to generate less of an inflammatory response, suggesting that synthetic linear genes may be used for gene therapy (Hofman et al. (2001) Gene Ther., 8:71-74). The synthetic linear genes may have an advantage over the circular plasmids in that most of the unwanted sequences that give inflammatory response is absent.

In genetic immunization, a technique used for vaccination and for antibody production, antigen-expressing plasmids are introduced into animals to elicit immune responses. Humoral and cellular immune responses to protein antigens can be efficiently primed by nucleic acid or DNA vaccination. In DNA-based vaccination, immunogenic proteins are expressed with correct post-translational modification, conformation, or oligomerization, thus ensuring the integrity of epitopes that stimulate neutralizing antibody (B-cell) responses. DNA (or RNA) immunization is a potent stimulator of T-cell responses because antigenic peptides are efficiently generated in endogenous or exogenous processing pathways, without interference by viral proteins, after transient in vivo transfection.

A vertebrate that is transgenic for a bacteriophage RNA polymerase, such as, but not limited to, T7 RNA polymerase, can be useful for producing antibodies by genetic immunization, or for protein expression studies such as in gene therapy. The ability to generate monoclonal antibodies using DNA-based immunizations dramatically reduces the time and resources needed to obtain research reagents that could aid in the identification, purification, and characterization of known or novel proteins. Use of a vertebrate that is transgenic for a bacteriophage RNA polymerase may eliminate the need of preparing plasmid DNA. For example, since T7 RNA polymerase transcribes RNA from linear DNA, a mouse transgenic for T7 RNA polymerase may be immunized with a single linear immunogenic construct, such as a PCR product or a pool of PCR products, and the humoral immune response (antibody production) can be measured by testing the sera.

This approach to antibody production can be adapted to a high-throughput format, saving time typically spent in cloning, production of protein antigens in a bacterial or insect cell system, and purification of the protein antigens. For example, cDNAs containing: (i) the promoter sequence cognate to the bacteriophage RNA polymerase, (ii) a eukaryotic ribosome recognition sequence, and iii) a sequence encoding the antigen, can be amplified by PCR in a 96-well format. The PCR products from individual wells can be injected into a mouse transgenic for the bacteriophage RNA polymerase. Alternatively, a pool of PCR products from different wells can be injected to a single mouse. Production of the resulting mouse antibodies can be screened against in vitro translated proteins coded by the PCR products. Each PCR product can serve as a template for synthesizing the corresponding antigen using coupled transcription and translation kits commercially available. This can provide enough protein to screen the sera from immunized mice. The synthesized protein can be coated on microtiter plate wells, or spotted onto immunoblotting membranes.

A vertebrate that is transgenic for a bacteriophage RNA polymerase, such as, but not limited to, T7 RNA polymerase, can also be used in protein expression studies or in identifying candidates for gene therapy. Co-transfection of plasmid expressing T7 RNA polymerase and a plasmid containing a gene downstream of a T7 RNA polymerase promoter can be used to express foreign genes in a mouse. This system has been shown to achieve rapid and high levels of gene expression in a variety of animal cells and tissues. In another example, a T7 cancer gene therapy plasmid vector, pT7T7/T7TK, was constructed to test the utility of the system in in vivo tumor inhibition (Chen et al. (1998) Hum. Gene Ther., 9:729-736). This nonviral vector contained a T7 autogene, T7T7, and a human herpes simplex virus thymidine kinase (HSV-TK) gene driven by a second T7 promoter (T7TK). When co-transfected with T7 RNA polymerase into cultured human osteosarcoma 143B cells, about 10-20% of the cells were found to express HSV-TK, and more than 90% of the cells were killed in the presence of 1 millimolar ganciclovir (GCV) within 4 days after DNA transfection. Direct injections of pT7T7/T7TK into 143B tumors grown in nude mice also resulted in TK gene expression in tumor cells located near the injection sites as revealed by the immunohistochemical staining. Repeated tumor injections of the pT7T7/T7TK vector and intraperitoneal injections of GCV resulted in inhibition of tumor growth and in tumor shrinkage in 6 out of 10 treated nude mice. These results, combined with the nonviral and rapid cytoplasmic gene expression features, suggest that the T7 RNA polymerase vector may be a good candidate for cancer gene therapy and other medical and biological applications. Vertebrates transgenic for T7 RNA polymerase (or other suitable bacteriophage RNA polymerase), such as a T7 RNA polymerase transgenic mouse, will eliminate the need for co-transfection of a target gene construct with a bacteriophage RNA polymerase expressing plasmid, thus eliminating the variable of differential expression of the bacteriophage RNA polymerase in vertebrate individuals.

The present invention provides a transgenic vertebrate whose genome includes a bacteriophage RNA polymerase as a transgene, wherein the bacteriophage RNA polymerase transgene is capable of being expressed in at least one cell of the transgenic vertebrate. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible.

The present invention also provides a method of expressing a protein in a transgenic vertebrate whose genome comprises a bacteriophage RNA polymerase transgene, including the steps of: a) providing a construct including the following elements operably linked: (i) a promoter sequence cognate to the bacteriophage RNA polymerase, (ii) a eukaryotic ribosome recognition sequence, and (iii) a sequence encoding the protein to be expressed; b) introducing the construct into at least one cell of the transgenic vertebrate; and c) providing conditions whereby the transgenic vertebrate expresses the protein. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The construct can further include any combination of the following: a stop codon, a tag sequence, or a poly-adenosine tail. The method can further include the step of isolating the protein. The present invention also claims the protein isolated by this method.

The present invention also provides a method to produce at least one antibody against an antigen in a transgenic vertebrate whose genome comprises a bacteriophage RNA polymerase transgene, including the steps of: a) providing a construct including the following elements operably linked: (i) a promoter sequence cognate to the bacteriophage RNA polymerase, (ii) a eukaryotic ribosome recognition sequence, and iii) a sequence encoding the antigen; b) introducing the construct into at least one cell of the transgenic vertebrate; and c) providing conditions whereby the transgenic vertebrate produces at least one antibody against the antigen. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The construct can further include any combination of the following: a stop codon, a tag sequence, or a poly-adenosine tail. The method can further include: (a) the step of isolating the at least one antibody as a polyclonal antibody; or (b) the steps of collecting spleen cells from the transgenic vertebrate, making at least one hybridoma from the spleen cells, and isolating the at least one antibody as a monoclonal antibody from the at least one hybridoma; or (c) the steps of collecting at least one egg from the bird and isolating at least one antibody as an IgY antibody from yolk of the at least one egg. The present invention further claims the at least one antibody produced by this method.

The present invention further provides a first method to produce a transgenic vertebrate whose genome includes a bacteriophage RNA polymerase transgene, including the steps of: a) introducing into the pronucleus of a fertilized ovum of a vertebrate a construct including a bacteriophage RNA polymerase as a transgene; b) transplanting the ovum into a female of the vertebrate; and c) allowing the ovum to develop to term, thereby producing a founder transgenic vertebrate individual. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The method can further include the step of breeding the founder transgenic vertebrate individual to obtain F1 transgenic vertebrates homozygous or hemizygous for said transgene.

The present invention further provides a second method to produce a transgenic vertebrate whose genome includes a bacteriophage RNA polymerase transgene, including the steps of: a) providing a transgene construct including a bacteriophage RNA polymerase as a transgene; b) introducing the transgene construct into embryonic stem cells of the vertebrate; c) selecting embryonic stem cells that have incorporated the transgene by recombination; d) introducing the embryonic stem cells that have incorporated the transgene by recombination into blastocysts of the vertebrate; e) transplanting the blastocysts into a pseudopregnant female of the vertebrate; and f) allowing the blastocysts to develop to term, thereby producing a chimeric founder transgenic vertebrate individual. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The recombination of the transgene may be homologous or heterologous. The method may further include the step of breeding the chimeric founder transgenic vertebrate individuals to obtain F1 transgenic vertebrates homozygous or hemizygous for said transgene. The transgene construct may further include a viral vector.

The present invention further provides a third method to produce a transgenic vertebrate whose genome includes a bacteriophage RNA polymerase transgene, including the steps of: a) providing a transgene construct including a bacteriophage RNA polymerase as a transgene; b) introducing the transgene construct into at least one embryonic cell of the vertebrate; c) selecting at least one embryonic cell that has incorporated the transgene by recombination; d) allowing the at least one embryonic cell that has incorporated the transgene by recombination to develop into at least one blastocyst of the vertebrate; e) transplanting the at least one blastocyst into a pseudopregnant female of the vertebrate; and f) allowing the at least one blastocyst to develop to term, thereby producing a chimeric founder transgenic vertebrate individual. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The recombination of the transgene may be homologous or heterologous. The method may further include the step of breeding the chimeric founder transgenic vertebrate individuals to obtain F1 transgenic vertebrates homozygous or hemizygous for said transgene. The transgene construct may further include a viral vector. The at least one embryonic cell of the vertebrate may be at least one morula cell.

The present invention further provides a fourth method to produce a transgenic vertebrate whose genome includes a bacteriophage RNA polymerase transgene, including the steps of: a) providing a transgene construct including a bacteriophage RNA polymerase as a transgene; b) introducing the transgene construct into at least one male germ-line stem cell of the vertebrate; c) selecting at least one male germ-line stem cell that has incorporated the transgene by recombination; d) introducing the at least one male germ-line stem cell that has incorporated the transgene by recombination into a recipient male of the vertebrate; e) allowing the at least one male germ-line stem cell that has incorporated the transgene by recombination to develop to maturity in the recipient male, thereby producing at least one mature transgenic spermatozoon; and f) breeding the recipient male carrying the at least one mature transgenic spermatozoon to obtain F1 transgenic vertebrates hemizygous for the transgene. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The recombination of the transgene may be homologous or heterologous. The method may further include the step of breeding the chimeric founder transgenic vertebrate individuals to obtain F1 transgenic vertebrates hemizygous for the transgene. The transgene construct may further include a viral vector.

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references.

Where a term is provided in the singular, the inventors also contemplate the plural of that term. The nomenclature used herein and the laboratory procedures described below are those well known and commonly employed in the art. Where there are discrepancies in terms and definitions used in references that are incorporated by reference, the terms used in this application shall have the definitions given herein. Other technical terms used herein have their ordinary meaning in the art that they are used, as exemplified by a variety of technical dictionaries (for example, Chambers Dictionary of Science and Technology, Peter M. B. Walker (editor), Chambers Harrap Publishers, Ltd., Edinburgh, UK, 1999, 1325 pp.). The inventors do not intend to be limited to a mechanism or mode of action. Reference thereto is provided for illustrative purposes only.

I. Transgenic Vertebrate

The present invention provides a transgenic vertebrate whose genome includes a bacteriophage RNA polymerase as a transgene, wherein the bacteriophage RNA polymerase transgene is capable of being expressed in at least one cell of the transgenic vertebrate. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible.

The transgenic vertebrate can be any vertebrate of interest, such as a mammal, a bird, a fish, a reptile, or an amphibian. Such vertebrates include vertebrates of economic or scientific interest. Suitable mammals include, but are not limited to, non-human primates, dogs, cats, sheep, pigs, goats, cattle, horses, rats, rabbits, and mice. Suitable birds include, but are not limited to, chickens, turkeys, ducks, and geese. Suitable fish include, but are not limited to, salmonids (for example, salmon and trout), cyprinids (for example, carp, goldfish, zebrafish), catfish, and tilapia. Suitable amphibians include, but are not limited to, anurans (for example, Rana spp. and Xenopus spp.) and urodeles (newts and salamanders).

The bacteriophage RNA polymerase can be any suitable bacteriophage RNA polymerase. Preferred bacteriophage RNA polymerases include T7 bacteriophage RNA polymerase, SP6 bacteriophage RNA polymerase, and T3 bacteriophage RNA polymerase. Particularly preferred is T7 bacteriophage RNA polymerase.

The bacteriophage RNA polymerase can be optionally linked to a promoter. The promoter can be any suitable promoter, and can be constitutive or inducible. Non-limiting examples of suitable constitutive promoters include SV40 promoter, CMV promoter, RSV promoter, CMV5 promoter-enhancer, actin promoter, and dihydrofolate reductase promoter. Non-limiting examples of inducible promoters include heat shock protein, metallothionien, human or mouse growth hormone, and drug-inducible promoters or enhancer elements (such as a tetracycline/doxycyclin-responsive element, an ecdysone-responsive element, or the lac repressor gene, lacI) (see, for example, Nordstrom (2002) Curr. Opin. Biotechnol., 13:453-458; and Fieck et al. (1992) Nucleic Acids Res., 20:1785-1791, which are incorporated by reference in their entirety herein). Promoters can be selected to preferentially express the bacteriophage RNA polymerase in a specific cell type or tissue. For example, a myosin light-chain promoter can be used to preferentially express the bacteriophage RNA polymerase in muscle, or a beta-lactoglobulin promoter can be used to preferentially express the bacteriophage RNA polymerase in mammary glands.

The transgenic vertebrate's genome includes the bacteriophage RNA polymerase as a transgene recombined into the vertebrate's genome. The bacteriophage RNA polymerase transgene is capable of being expressed in at least one cell of the transgenic vertebrate, more preferably capable of being expressed in at least one type of cell (such as, but not limited to, blood cells, spleen cells, and skin cells) or at least one type of tissue (such as, but not limited to, brain and other nervous tissue, muscle, and liver) of the transgenic vertebrate. In some cases, the bacteriophage RNA polymerase transgene is expressed throughout the body of the transgenic vertebrate. Expression can be constitutive when the bacteriophage RNA polymerase transgene is under the control of a constitutive promoter. Alternatively, expression can be induced when the bacteriophage RNA polymerase transgene is under the control of an inducible promoter.

II. Method of Expressing a Protein in a Transgenic Vertebrate

The present invention also provides a method of expressing a protein in a transgenic vertebrate whose genome comprises a bacteriophage RNA polymerase transgene, including the steps of: a) providing a construct including the following elements operably linked: (i) a promoter sequence cognate to the bacteriophage RNA polymerase, (ii) a eukaryotic ribosome recognition sequence, and (iii) a sequence encoding the protein to be expressed; b) introducing the construct into at least one cell of the transgenic vertebrate; and c) providing conditions whereby the transgenic vertebrate expresses the protein. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The construct can further include any combination of the following: a stop codon, a tag sequence, or a poly-adenosine tail. The method can further include the step of isolating the protein. The present invention also claims the protein isolated by this method.

Constructs for use in the method of the invention include the following elements operably linked: a promoter sequence cognate to the bacteriophage RNA polymerase transgene, a eukaryotic ribosome recognition site, and a sequence encoding the protein to be expressed. A non-limiting example of a cognate promoter sequence is the T7 RNA polymerase promoter (Rosa (1979) Cell, 16:815-825, which is incorporated by reference in its entirety herein), where the bacteriophage RNA polymerase transgene is T7 RNA polymerase. The eukaryotic ribosome recognition site is preferably, but not necessarily, a Kozak sequence (see “Extracting Kozak Consensus Sequence Using Kleisli”, Chen et al., available on-line at www.bionet.nsc.ru/bgrs/thesis/61/, accessed 28 Oct. 2003; Kozak (1982) Biochem. Soc. Symp., 47:113-128; Kozak (1982) J. Virol., 42:467-473, and Kozak (1987) Nucleic Acids Res., 15:8125-8148, which are incorporated by reference in their entirety herein). An internal ribosome entry site (IRES) (Hellen and Sarnow (2001) Genes & Dev., 15:1593-1612; Vagner et al. (2001) EMBO Rep., 2:893-898; Martinez-Salas (1999) Curr. Opin. Biotechnol., 10:458-464; and Mountford and Smith (1995) Trends Genet., 11:179-184, which are incorporated by reference in their entirety herein) can function as a eukaryotic ribosome recognition site. The protein to be expressed can be any suitable protein, including naturally occurring proteins and their homologues, fusion proteins, hormones, enzymes, polypeptides, antibodies, antibody fragments, antigens, or epitopes.

The construct optionally includes any combination of a stop codon, a tag sequence, and a poly-adenosine tail. The construct may be linear or circular. Circular constructs preferably include a stop codon. Linear constructs need not include a stop codon. Constructs may include a tag sequence in frame useful in purification of the protein (for example, by affinity purification) or for confirmation of protein production (for example, via Western blot analysis). Non-limiting examples of suitable tag sequences include a poly-histidine sequence, a FLAG® sequence, c-myc, glutathione S-transferase (GST), hemagglutinin (HA), and other epitopes (Pati (1992) Gene, 114:285-288; Cravchik and Matus (1993) Gene, 137:139-143; and Nakajima and Yaoita (1997) Nucleic Acids Res., 25:2231-2232, which are incorporated by reference in their entirety herein). Where increased transcript stability is desired, constructs preferably include a poly-adenosine tail.

Protein sequences to be expressed, are obtainable from many sources. For example, nucleic acid sequences and cDNA sequences are readily available at a variety of Internet Web sites (for example, GenBank and other databases at the National Center for Biotechnology Information, accessible on-line at www.ncbi.nlm.nih.gov, and the Swiss-Prot database, accessible on-line at us.expasy.org/sprot/sprot-top.html). Oligonucleotides can designed based on these sequences and synthesized by one of several industrial suppliers. In another example, linear transcription constructs can be generated from individual genes or from genomic DNA (in case of microbial genome and some intron-less genes) to create individual or libraries of templates using specially designed primers and subsequently used for protein expression in vivo (Sykes and Johnston (1999) Nature Biotechnol., 17:355-359).

Constructs for use in the method of the invention may be introduced into at least one cell of the transgenic vertebrate by any suitable means. The construct may be introduced as a DNA vaccine (see, for example, U.S. Pat. No. 6,413,942 to Feigner et al., “Methods of delivering a physiologically active polypeptide to a mammal”, issued 2 Jul. 2002; U.S. Pat. No. 6,384,018 to Content et al., “Polynucleotide tuberculosis vaccine”, issued 7 May 2002; U.S. Pat. No. 6,214,804 to Feigner et al., “Induction of a protective immune response in a mammal by injecting a DNA sequence”, issued 10 Apr. 2001; U.S. Pat. No. 5,846,946 to Huebner et al., “Compositions and methods for administering Borrelia DNA”, issued 8 Dec. 1998; U.S. Pat. No. 5,703,055 to Feigner et al., “Generation of antibodies through lipid mediated DNA delivery”, issued 30 Dec. 1997; U.S. Pat. No. 5,589,466 to Feigner et al., “Induction of a protective immune response in a mammal by injecting a DNA sequence”, issued 31 Dec. 1996; Hasan et al. (1999) J. Immunol. Methods, 229:1-22; Lewis and Babiuk (1999) Adv. Virus Res., 54:129-188; and Gurunathan et al. (2000) Annu. Rev. Immunol., 18:927-974, which are incorporated by reference in their entirety herein). The construct may be introduced as an RNA vaccine (see, for example, McKenzie et al., (2001) Immunol. Res., 24:225-244; Hoerr et al. (2000) Eur. J. Immunol., 30:1-7; and Ying et al. (1999) Nature Med., 5:823-827, which are incorporated by reference in their entirety herein). The construct can be a single-stranded polynucleotide or double-stranded polynucleotide or any combination of both. The construct can be a naked polynucleotide or a complexed polynucleotide (Pachuk et al. (2000) Curr. Opin. Mol. Ther., 2:188-198; and Hoerr et al. (2000) Eur. J. Immunol., 30:1-7, which are incorporated by reference in their entirety herein).

The construct can include a suitable vector, such as a mammalian expression vector or a viral vector. Suitable vectors include, but are not limited to, a phage, cosmid, retrovirus, vaccinia, adenovirus, adeno-associated virus, herpes simplex virus, papillomavirus, Epstein Barr virus (EBV), and the like (U.S. Pat. No. 5,910,488 to Nabel et al., “Plasmids suitable for gene therapy”, issued 8 Jun. 1999, which is incorporated by reference in its entirety herein). Preferably, the vector is defective in that it lacks functional virulence genes, such that it is not infective after introduction into the target cell (Zeng et al. (1998) Cell Biol. Toxicol., 14:105-110; Somia et al. (1999) Nature Biotechnol., 17:224-225; Stratford-Perricaudet et al. (1992) J. Clin. Invest., 90:626-630; Samulski et al. (1989) J. Virol., 63:3822-3828; and Kaplitt and Makimura (1991) J. Neurosci. Methods, 71:125-32, which are incorporated by reference in their entirety herein). Alternatively, constructs of the present invention may be introduced by lipofection in vivo using liposomes (Felgner et al. (1987) Proc. Natl. Acad. Sci. USA, 84:7413-7417, which is incorporated by reference in its entirety herein). Liposomes may be targeted to particular tissues or cell types by coupling lipids to other molecules, for example, receptor ligands or antibodies that bind to a particular cell type. Constructs, in vectors or not in vectors, may also be applied to or formulated within a matrix, such as a polymeric solid matrix, a semisolid or gel, or a membrane, which is introduced into or applied externally to the mammal or cell to be treated.

Constructs may be designed for studies of protein expression or for gene therapy in a vertebrate. Such constructs may be in viral vectors, in liposomes, or not in a vector. Constructs may be also be introduced into cells ex vivo. Cells of the vertebrate (such as, but not limited to, circulating plasma cells, ova, or spermatogenic cells) or tissues (such as, but not limited to, liver or bone marrow) may be removed from the body. The constructs, in or not in a vector, may be introduced into the cells ex vivo by any appropriate method, such as by infection, as a calcium phosphate precipitate, or by lipofection, electroporation, or other methods known or developed in the art. After introduction of the construct into the cells, the cells can be reintroduced into the body of the vertebrate, or into another vertebrate.

The compositions used in introducing the construct into at least one cell of a vertebrate can include, in addition to the construct, at least one co-stimulatory factor (Frauwirth and Thompson (2002) J. Clin. Invest., 109:295-299, which is incorporated by reference in its entirety herein). Suitable co-stimulatory factors include, for example, such molecules as B7 and CD40, cytokines, mitogens, antibodies, antigen-presenting cells (Mayordomo et al. (1997) Stem Cells, 15:94-103, which is incorporated by reference in its entirety herein), and peptides derived from a helper T-lymphocyte epitope foreign to the immunized mammal. Co-stimulatory factors can be delivered together with the construct used for immunization, for example as a fusion with the construct, or separately, for example as a separate peptide or a separate nucleic acid molecule encoding a peptide. These co-stimulatory factors can be delivered as genes, for example, as genes for a co-stimulatory cytokine or other co-stimulatory factor (Scheerlinck (2001) Vaccine, 19:2647-56; and Cohen et al. (1998) FASEB J., 12:1611-1626, which are incorporated by reference in their entirety herein).

Conditions whereby the transgenic vertebrate expresses the protein are provided as necessary or desired. Such conditions include, but are not limited to, introduction of the appropriate inducer molecule (such as a drug) or condition (such as a particular temperature) where the bacteriophage RNA polymerase transgene is under the control of an inducible promoter; immunization of the transgenic vertebrate with the construct under an immunization schedule appropriate for the vertebrate species; or use of co-stimulatory factors (Frauwirth and Thompson (2002) J. Clin. Invest., 109:295-299).

Proteins expressed by the method of the invention can be optionally isolated or purified to the degree of purity desired by methods known in the art. See, for example, “Protein Purification: Principles and Practice”, R. K. Scopes, Springer Verlag, 1994, 3rd edition, 380 pp.; “Cloning, Gene Expression and Protein Purification: Experimental Procedures and Process Rationale”, Hardin et al., editors, Oxford University Press, 2001, 448 pp.; and “Protein Protocols Handbook”, J. M. Walker, editor, Human Press, 2002, 2nd edition, 1176 pp., which are incorporated by reference in their entirety herein.

III. Method of Producing an Antibody in a Transgenic Vertebrate

The present invention also provides a method to produce at least one antibody against an antigen in a transgenic vertebrate whose genome comprises a bacteriophage RNA polymerase transgene, including the steps of: a) providing an immunogenic construct including the following elements operably linked: (i) a promoter sequence cognate to the bacteriophage RNA polymerase, (ii) a eukaryotic ribosome recognition sequence, and iii) a sequence encoding the antigen; b) introducing the immunogenic construct into at least one cell of the transgenic vertebrate; and c) providing conditions whereby the transgenic vertebrate produces at least one antibody against the antigen. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The immunogenic construct can further include any combination of the following: a stop codon, a tag sequence, or a poly-adenosine tail. The method can further include: (a) the step of isolating the at least one antibody as a polyclonal antibody; or (b) the steps of collecting spleen cells from the transgenic vertebrate, making at least one hybridoma from the spleen cells, and isolating the at least one antibody as a monoclonal antibody from the at least one hybridoma; or (c) the steps of collecting at least one egg from the bird and isolating at least one antibody as an IgY antibody from yolk of the at least one egg. The present invention further claims the at least one antibody produced by this method.

Immunogenic constructs for use in the method of the invention include the following elements operably linked: a promoter sequence cognate to the bacteriophage RNA polymerase transgene, a eukaryotic ribosome recognition site, and a sequence encoding the antigen to be expressed. A non-limiting example of a cognate promoter sequence is the T7 RNA polymerase promoter, where the bacteriophage RNA polymerase transgene is T7 RNA polymerase. A non-limiting example of a cognate promoter sequence is the T7 RNA polymerase promoter (Rosa (1979) Cell, 16:815-825, which is incorporated by reference in its entirety herein), where the bacteriophage RNA polymerase transgene is T7 RNA polymerase. The eukaryotic ribosome recognition site is preferably, but not necessarily, a Kozak sequence (see “Extracting Kozak Consensus Sequence Using Kleisli”, Chen et al., available on-line at www.bionet.nsc.rulbgrs/thesis/61/, accessed 28 Oct. 2003; Kozak (1982) Biochem. Soc. Symp., 47:113-128; Kozak (1982) J. Virol., 42:467-473, and Kozak (1987) Nucleic Acids Res., 15:8125-8148, which are incorporated by reference in their entirety herein). An internal ribosome entry site (IRES) (Hellen and Sarnow (2001) Genes & Dev., 15:1593-1612; Vagner et al. (2001) EMBO Rep., 2:893-898; Martinez-Salas (1999) Curr. Opin. Biotechnol., 10:458-464; and Mountford and Smith (1995) Trends Genet., 11:179-184, which are incorporated by reference in their entirety herein) can function as a eukaryotic ribosome recognition site. The antigen to be expressed can be any suitable antigen, including naturally occurring proteins and their homologues, fusion proteins, polypeptides, and epitopes.

The immunogenic construct optionally includes any combination of a stop codon, a tag sequence, and a poly-adenosine tail. The immunogenic construct may be linear or circular. Circular immunogenic constructs preferably include a stop codon. Linear immunogenic constructs need not include a stop codon. Immunogenic constructs may include a tag sequence in frame useful in purification of the protein (for example, by affinity purification) or for confirmation of protein production (for example, via Western blot analysis). Non-limiting examples of suitable tag sequences include a poly-histidine sequence, a FLAG® sequence, c-myc, glutathione S-transferase (GST), hemagglutinin (HA), and other epitopes (see, for example, Pati (1992) Gene, 114:285-288; Cravchik and Matus (1993) Gene, 137:139-143; and Nakajima and Yaoita (1997) Nucleic Acids Res., 25:2231-2232, which are incorporated by reference in their entirety herein). Where increased transcript stability is desired, immunogenic constructs preferably include a poly-adenosine tail.

Antigen sequences to be expressed, are obtainable from many sources. For example, nucleic acid sequences and cDNA sequences are readily available at a variety of Internet Web sites (for example, GenBank and other databases at the National Center for Biotechnology Information, accessible on-line at www.ncbi.nlm.nih.gov, and the Swiss-Prot database, accessible on-line at us.expasy.org/sprot/sprot-top.html). Oligonucleotides can designed based on these sequences and synthesized by one of several industrial suppliers. In another example, linear transcription immunogenic constructs can be generated from individual genes or from genomic DNA (in case of microbial genome and some intron-less genes) to create individual or libraries of templates using specially designed primers and subsequently used for protein expression in vivo (Sykes and Johnston (1999) Nature Biotechnol., 17:355-359).

Immunogenic constructs for use in the method of the invention may be introduced into at least one cell of the transgenic vertebrate by any suitable means, such as a DNA vaccine or as an RNA vaccine, as a single-stranded polynucleotide or double-stranded polynucleotide or any combination of both, as a naked polynucleotide or a complexed polynucleotide, in a vector or not in a vector (as described above under the heading “Method of Expressing a Protein in a Transgenic Vertebrate”). Immunogenic constructs may be introduced by methods as described above under the heading “Method of Expressing a Protein in a Transgenic Vertebrate”, including by lipofection in vivo; by the use of a matrix, a semisolid or gel, or a membrane, which is introduced into or applied externally to the mammal or cell to be treated; or by introduction to cells or tissues of the vertebrate ex vivo.

The compositions used in introducing the immunogenic construct into at least one cell of a vertebrate can include, in addition to the immunogenic construct, at least one co-stimulatory factor (Frauwirth and Thompson (2002) J. Clin. Invest., 109:295-299, which is incorporated in its entirety herein). Suitable co-stimulatory factors include, for example, such molecules as B7 and CD40, cytokines, mitogens, antibodies, antigen-presenting cells (Mayordomo et al. (1997) Stem Cells, 15:94-103, which is incorporated in its entirety herein), and peptides derived from a helper T-lymphocyte epitope foreign to the immunized vertebrate. Co-stimulatory factors can be delivered together with the immunogenic construct used for immunization, for example as a fusion with the immunogenic construct, or separately, for example as a separate peptide or a separate nucleic acid molecule encoding a peptide. These co-stimulatory factors can be delivered as genes, for example, as genes for a co-stimulatory cytokine or other co-stimulatory factor (Scheerlinck (2001) Vaccine, 19:2647-56; and Cohen et al. (1998) FASEB J., 12:1611-1626, which are incorporated by reference in their entirety herein).

Conditions whereby the transgenic vertebrate produces at least one antibody against the antigen are provided as necessary or desired. Such conditions include, but are not limited to, introduction of the appropriate inducer molecule (such as a drug) or condition (such as a particular temperature) where the bacteriophage RNA polymerase transgene is under the control of an inducible promoter; immunization of the transgenic vertebrate with the immunogenic construct under an immunization schedule appropriate for the vertebrate species; or use of co-stimulatory factors (Frauwirth and Thompson (2002) J. Clin. Invest., 109:295-299).

The method can further include steps to isolate or purify the antibody produced by the transgenic vertebrate. Such steps are well known in the art and include: (a) the step of isolating the at least one antibody as a polyclonal antibody; or (b) the steps of collecting spleen cells from the transgenic vertebrate, making at least one hybridoma from the spleen cells, and isolating the at least one antibody as a monoclonal antibody from the at least one hybridoma; or (c) the steps of collecting at least one egg from the bird and isolating at least one antibody as an IgY antibody from yolk of the at least one egg (see, for example, “Antibodies: A Laboratory Manual”, E. Harlow and D. Lane, editors, Cold Spring Harbor Laboratory, 1988, 726 pp; “Monoclonal Antibodies: A Practical Approach”, P. Shepherd and C. Dean, editors, Oxford University Press, 2000, 479 pp.; “Chicken Egg Yolk Antibodies, Production and Application: IgY-Technology (Springer Lab Manual)”, by R Schade et al., editors, Springer-Verlag, 2001, 255 pp., which are incorporated by reference in their entirety herein). The present invention further claims the at least one antibody, polyclonal or monoclonal or IgY, produced by this method.

IV. Methods to Produce a Transgenic Vertebrate

The present invention further provides methods to produce a transgenic vertebrate whose genome includes a bacteriophage RNA polymerase transgene, as described above under the heading “Transgenic Vertebrate”. The transgenic vertebrate can be any vertebrate of interest, such as a mammal, a bird, a fish, a reptile, or an amphibian, and including vertebrates of economic or scientific interest. The bacteriophage RNA polymerase can be any suitable bacteriophage RNA polymerase. Preferred bacteriophage RNA polymerases include T7 bacteriophage RNA polymerase, SP6 bacteriophage RNA polymerase, and T3 bacteriophage RNA polymerase. Particularly preferred is T7 bacteriophage RNA polymerase. The bacteriophage RNA polymerase can be optionally linked to a promoter. The promoter can be any suitable promoter, and can be constitutive or inducible. Promoters can be selected to preferentially express the bacteriophage RNA polymerase in a specific cell type or tissue.

Constructs for use in methods of the invention include the bacteriophage RNA polymerase as a transgene, optionally linked to a promoter. Such constructs can be made by methods known in the art (Molecular Cloning: A Laboratory Manual, Joseph Sambrook et al., Cold Spring Harbor Laboratory, 2001, 999 pp.; Short Protocols in Molecular Biology, Frederick M. Ausubel et al. (editors), John Wiley & Sons, 2002, 1548 pp.), such as by expression in a plasmid or other vector. The bacteriophage RNA polymerase gene can be obtained by any suitable means, such as by cloning from a bacterium or other organism that expresses the gene, or from the bacteriophage. Sequences of the bacteriophage RNA polymerase gene are available, for example from GenBank and other databases at the National Center for Biotechnology Information, accessible on-line at www.ncbi.nlm.nih.gov, and appropriate sequence-specific oligonucleotide primers can be designed using the gene sequence information. The nucleotide sequence of the T7 RNA polymerase gene has been published (Moffatt et al. (1984) J. Mol. Biol., 173:265-269; and U.S. Pat. No. 4,952,496 to Studier et al., “Cloning and expression of the gene for bacteriophage T7 RNA polymerase”, issued 28 Aug. 1990, which are incorporated by reference in their entirety herein).

The transgenic vertebrate's genome includes the bacteriophage RNA polymerase as a transgene recombined into the vertebrate's genome, as described above under the heading “Transgenic Vertebrate”. The bacteriophage RNA polymerase transgene is capable of being expressed in at least one cell of the transgenic vertebrate, more preferably capable of being expressed in at least one type of cell or at least one type of tissue of the transgenic vertebrate. In some cases, the bacteriophage RNA polymerase transgene is expressed throughout the body of the transgenic vertebrate. Expression can be constitutive when the bacteriophage RNA polymerase transgene is under the control of a constitutive promoter. Alternatively, expression can be induced when the bacteriophage RNA polymerase transgene is under the control of an inducible promoter.

Methods to produce transgenic vertebrates are known in the art. See, for example, “Transgenic Animal Technology: A Laboratory Handbook”, C. A. Pinkert, editor, Academic Press, 2002, 2nd edition, 618 pp.; “Mouse Genetics and Transgenics: A Practical Approach”, I. J. Jackson and C. M. Abbott, editors, Oxford University Press, 2000, 299 pp.; “Transgenesis Techniques: Principles and Protocols”, A. R. Clarke, editor, Humana Press, 2001, 351 pp.; “Animal Breeding: Technology for the 21st Century”, A. J. Clark, editor, Taylor & Francis, 1998, 268 pp.; Houdebine (2002) J. Biotechnol., 98:145-160 Wolfgang and Golos (2002) Trends Biotechnol., 20:479-484; Chan et al. (2001) Science, 291:309-312; Bode et al. (2000) Biol. Chem., 381:801-813; Pintado and Guitierrez-Adan (1999) Reprod. Nutr. Dev., 39:535-544; Rudolph (1999) Trends Biotechnol., 17:367-374; Mullins and Mullins (1996) J. Clin. Invest., 97:1557-1560; Perry and Sang (1993) Transgenic Res., 2:125-133; and Houdebine and Chourrout (1991) Experientia, 47:891-897, which are incorporated by reference in their entirety herein. Non-limiting examples of methods to produce an transgenic vertebrate whose genome includes a bacteriophage RNA polymerase transgene are described below.

A. A First Method to Produce a Transgenic Vertebrate

The present invention further provides a first method to produce a transgenic vertebrate whose genome includes a bacteriophage RNA polymerase transgene, including the steps of: a) introducing into the pronucleus of a fertilized ovum of a vertebrate a construct including a bacteriophage RNA polymerase as a transgene; b) transplanting the ovum into a female of the vertebrate; and c) allowing the ovum to develop to term, thereby producing a founder transgenic vertebrate individual. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The method can further include the step of breeding the founder transgenic vertebrate individual to obtain F1 transgenic vertebrates.

The construct containing the bacteriophage RNA polymerase as a transgene, optionally linked to a promoter, is introduced into the pronucleus of a fertilized ovum of a vertebrate (for example, a mouse). See, for example, “Transgenic Animal Technology: A Laboratory Handbook”, C. A. Pinkert, editor, Academic Press, 2002, 2nd edition, 618 pp.; “Mouse Genetics and Transgenics: A Practical Approach”, I. J. Jackson and C. M. Abbott, editors, Oxford University Press, 2000, 299 pp.; Misra and Duncan (2002) Endocrine, 19:229-238; Hammer et al. (1986) J. Anim. Sci., 63:269-278; and Palmiter and Brinster (1986) Ann. Rev. Genetics, 20:465-499, which are incorporated by reference in their entirety herein. Transgenic ova are transplanted into recipient pseudopregnant females of the vertebrate species and allowed to develop to term to produce founder transgenic vertebrate individuals. Screening to ensure incorporation of the transgene into the genome may be carried out on the ova or on the resulting term offspring. Founder transgenic vertebrate individuals may be further bred, to each other or to wild-type individuals, to obtain homozygous or hemizygous F1 transgenic vertebrates.

B. A Second Method To Produce A Transgenic Vertebrate

The present invention further provides a second method to produce a transgenic vertebrate whose genome includes a bacteriophage RNA polymerase transgene, including the steps of: a) providing a transgene construct including a bacteriophage RNA polymerase as a transgene; b) introducing the transgene construct into embryonic stem cells of the vertebrate; c) selecting embryonic stem cells that have incorporated the transgene by recombination; d) introducing the embryonic stem cells that have incorporated the transgene by recombination into blastocysts of the vertebrate; e) transplanting the blastocysts into a pseudopregnant female of the vertebrate; and f) allowing the blastocysts to develop to term, thereby producing a chimeric founder transgenic vertebrate individual. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The method may further include the step of breeding the chimeric founder transgenic vertebrate individuals to obtain F1 transgenic vertebrates hemizygous for the transgene. The transgene construct may further include a viral vector.

The construct containing the bacteriophage RNA polymerase as a transgene, optionally linked to a promoter, may optionally include a viral vector. The viral vector can be any suitable viral vector, such as, but not limited to, a lentivirus, a retrovirus, an adenovirus, an adeno-associated virus, and other viral vectors including replication-deficient viruses and hybrid viral vectors. See, for example, Pfeifer et al. (2002), Proc. Natl. Acad. Sci. USA, 99:2140-2145; Lois et al. (2002) Science, 295:868-872; Rubinson et al. (2003), Nature Genetics, 33:401-406; Harvey et al. (2003) Poult. Sci., 82:927-930; Briskin et al. (1991) Proc. Natl. Acad. Sci. USA, 88:1736-1740; Linney et al. (1999) Dev. Biol., 213:207-216; Ghazizadeh et al. (1997) J. Virol., 71: 9163-9169; Hollenbeck and Fekete (2003) Methods Cell Biol., 71:369:386; Morsy et al. (1998) Proc. Natl. Acad. Sci. USA, 95:7866-7871, Ohnishi et al. (2002) Gene Ther., 9:303-306; Ketteler et al. (2002) Gene Ther., 9:477-487; and Lam and Breakefield (2000) J. Gene. Med., 2:395-408, which are incorporated by reference in their entirety herein.

Methods for introducing trangenes into embryonic stem cells are known in the art. See, for example, “Transgenic Animal Technology: A Laboratory Handbook”, C. A. Pinkert, editor, Academic Press, 2002, 2nd edition, 618 pp.; “Mouse Genetics and Transgenics: A Practical Approach”, I. J. Jackson and C. M. Abbott, editors, Oxford University Press, 2000, 299 pp.; and “Transgenesis Techniques: Principles and Protocols”, A. R. Clarke, editor, Humana Press, 2001, 351 pp., which are incorporated by reference in their entirety herein. The construct is introduced into embryonic stem cells of the vertebrate (for example, a mouse). Embryonic stem cells that have incorporated the transgene by recombination are selected and introduced into blastocysts of the vertebrate; the chimeric blastocysts are transplanted into a pseudopregnant female of the vertebrate (such as a mouse) and allowed to develop to term, thereby producing a chimeric founder transgenic vertebrate individual. The resulting chimeric founder transgenic vertebrate individuals may be optionally bred, to each other or to wild-type individuals, to obtain homozygous or hemizygous F1 transgenic vertebrates.

C. A Third Method to Produce a Transgenic Vertebrate

The present invention further provides a third method to produce a transgenic vertebrate whose genome includes a bacteriophage RNA polymerase transgene, including the steps of: a) providing a transgene construct including a bacteriophage RNA polymerase as a transgene; b) introducing the transgene construct into at least one embryonic cell of the vertebrate; c) selecting at least one embryonic cell that has incorporated the transgene by recombination; d) allowing the at least one embryonic cell that has incorporated the transgene by recombination to develop into at least one blastocyst of the vertebrate; e) transplanting the at least one blastocyst into a pseudopregnant female of the vertebrate; and f) allowing the at least one blastocyst to develop to term, thereby producing a chimeric founder transgenic vertebrate individual. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The recombination of the transgene may be homologous or heterologous. The method may further include the step of breeding the chimeric founder transgenic vertebrate individuals to obtain F1 transgenic vertebrates hemizygous for the transgene. The transgene construct may further include a viral vector. The at least one embryonic cell of the vertebrate may be at least one morula cell.

The construct containing the bacteriophage RNA polymerase as a transgene, optionally linked to a promoter, may optionally include a viral vector. The viral vector can be any suitable viral vector, such as, but not limited to, a lentivirus, a retrovirus, an adenovirus, an adeno-associated virus, and other viral vectors including replication-deficient viruses and hybrid viral vectors. See, for example, Pfeifer et al. (2002), Proc. Natl. Acad. Sci. USA, 99:2140-2145; Lois et al. (2002) Science, 295:868-872; Rubinson et al. (2003), Nature Genetics, 33:401-406; Harvey et al. (2003) Poult. Sci., 82:927-930; Briskin et al. (1991) Proc. Natl. Acad. Sci. USA, 88:1736-1740; Linney et al. (1999) Dev. Biol., 213:207-216; Ghazizadeh et al. (1997) J. Virol., 71: 9163-9169; Hollenbeck and Fekete (2003) Methods Cell Biol., 71:369:386; Morsy et al. (1998) Proc. Natl. Acad. Sci. USA, 95:7866-7871; Ohnishi et al. (2002) Gene Ther., 9:303-306; Ketteler et al. (2002) Gene Ther., 9:477-487; and Lam and Breakefield (2000) J. Gene. Med., 2:395-408, which are incorporated by reference in their entirety herein.

Methods for introducing trangenes into embryonic cells are known in the art. See, for example, “Transgenic Animal Technology: A Laboratory Handbook”, C. A. Pinkert, editor, Academic Press, 2002, 2nd edition, 618 pp.; “Mouse Genetics and Transgenics: A Practical Approach”, I. J. Jackson and C. M. Abbott, editors, Oxford University Press, 2000, 299 pp.; “Transgenesis Techniques: Principles and Protocols”, A. R. Clarke, editor, Humana Press, 2001, 351 pp., Briskin et al. (1991) Proc. Natl. Acad. Sci. USA, 88:1736-1740; Pfeifer et al. (2002), Proc. Natl. Acad. Sci. USA, 99:2140-2145; and Houdebine and Chourrout (1991) Experientia, 47:891-897, which are incorporated by reference in their entirety herein. The construct is introduced into at least one embryonic cell of the vertebrate (for example, at least one preimplantation embryonic cell of a mouse or at least one blastocyst of a chicken). The at least one embryonic cell of the vertebrate may be any suitable embryonic cell, including a zygote. The at least one embryonic cell of the vertebrate is preferably at least one preimplantation embryonic cell, such as at least one pluripotent embryonic stem cell, at least one blastocyst cell, or at least one morula cell. At least one embryonic cell that has incorporated the transgene by recombination is selected and allowed to develop into at least one blastocyst of the vertebrate, which is transplanted into a pseudopregnant female of the vertebrate (such as a pseudopregnant female mouse or a hen) and allowed to develop to term, thereby producing a chimeric founder transgenic vertebrate individual. The resulting chimeric founder transgenic vertebrate individuals may be optionally bred, to each other or to wild-type individuals, to obtain homozygous or hemizygous F1 transgenic vertebrates.

D. A Fourth Method to Produce a Transgenic Vertebrate

The present invention further provides a fourth method to produce a transgenic vertebrate whose genome includes a bacteriophage RNA polymerase transgene, including the steps of: a) providing a transgene construct including a bacteriophage RNA polymerase as a transgene; b) introducing the transgene construct into at least one male germ-line stem cell of the vertebrate; c) selecting at least one male germ-line stem cell that has incorporated the transgene by recombination; d) introducing the at least one male germ-line stem cell that has incorporated the transgene by recombination into a recipient male of the vertebrate; e) allowing the at least one male germ-line stem cell that has incorporated the transgene by recombination to develop to maturity in the recipient male, thereby producing at least one mature transgenic spermatozoon; and f) breeding the recipient male carrying the at least one mature transgenic spermatozoon to obtain F1 transgenic vertebrates hemizygous for the transgene. The transgenic vertebrate can be a bird, a fish, an amphibian, or a mammal. The bacteriophage RNA polymerase can be a T7, a SP6, or a T3 RNA polymerase, and can be optionally linked to a promoter, either constitutive or inducible. The recombination of the transgene may be homologous or heterologous. The method may further include the step of breeding the chimeric founder transgenic vertebrate individuals to obtain F1 transgenic vertebrates hemizygous for the transgene. The transgene construct may further include a viral vector.

The construct containing the bacteriophage RNA polymerase as a transgene, optionally linked to a promoter, may optionally include a viral vector. The viral vector can be any suitable viral vector, such as, but not limited to, a lentivirus, a retrovirus, an adenovirus, an adeno-associated virus, and other viral vectors including replication-deficient viruses and hybrid viral vectors. See, for example, Hamra et al. (2002) Proc. Natl. Acad. Sci. USA, 99:14931-14936; Pfeifer et al. (2002), Proc. Natl. Acad. Sci. USA, 99:2140-2145; Lois et al. (2002) Science, 295:868-872; Rubinson et al. (2003), Nature Genetics, 33:401-406; Harvey et al. (2003) Poult. Sci., 82:927-930; Briskin et al. (1991) Proc. Natl. Acad. Sci. USA, 88:1736-1740; Linney et al. (1999) Dev. Biol., 213:207-216; Ghazizadeh et al. (1997) J. Virol., 71: 9163-9169; Hollenbeck and Fekete (2003) Methods Cell Biol., 71:369:386; Morsy et al. (1998) Proc. Natl. Acad. Sci. USA, 95:7866-7871; Ohnishi et al. (2002) Gene Ther., 9:303-306; Ketteler et al. (2002) Gene Ther., 9:477-487; and Lam and Breakefield (2000) J. Gene. Med., 2:395-408, which are incorporated by reference in their entirety herein.

Methods for introducing transgenes into male germ-line stem cells are known in the art. See, for example, “Transgenic Animal Technology: A Laboratory Handbook”, C. A. Pinkert, editor, Academic Press, 2002, 2nd edition, 618 pp.; “Mouse Genetics and Transgenics: A Practical Approach”, I. J. Jackson and C. M. Abbott, editors, Oxford University Press, 2000, 299 pp.; “Transgenesis Techniques: Principles and Protocols”, A. R. Clarke, editor, Humana Press, 2001, 351 pp., Hamra et al. (2002) Proc. Natl. Acad. Sci. USA, 99:14931-14936; Nagano et al. (2001) Proc. Natl. Acad. Sci. USA, 98: 13090-13095; and Pintado and Guitierrez-Adan (1999) Reprod. Nutr. Dev., 39:535-544; which are incorporated by reference in their entirety herein. The transgene construct is introduced into at least one male germ-line stem cell of the vertebrate. The at least one male germ-line stem cell may be any suitable male germ-line stem cell that is capable of developing into a mature spermatozoon, including a gonocyte or a spermatogonial stem cell (see McLean et al. (2003), Biol. Reprod., Papers in Press, published online ahead of print Sep. 3, 2003 and available at www.biolreprod.org/cgi/rapidpdf/biolreprod. 103.017020v1.pdf, accessed 28 Oct. 2003). At least one male germ-line stem cell that has incorporated the transgene by recombination is selected and introduced into a recipient male of the vertebrate (for example, by transplantation into the seminiferous tubules of a recipient male mouse or rat), where it is allowed to develop to maturity in the recipient male, thereby producing at least one mature transgenic spermatozoon. The recipient male carrying the at least one mature transgenic spermatozoon can be bred with wild-type vertebrate individuals to obtain F1 transgenic vertebrates hemizygous for the transgene.

### EXAMPLES

### Example 1

### Production of a Transgenic Mouse Expressing T7 RNA Polymerase

The following non-limiting example describes the production of a transgenic vertebrate whose genome includes a bacteriophage RNA polymerase, specifically a mouse strain expressing T7 RNA polymerase (T7 RNAP). This example describes construction of a plasmid DNA vector where T7 RNAP is constitutively expressed under the control of a CMV promoter, and microinjection of the DNA into a fertilized mouse egg.

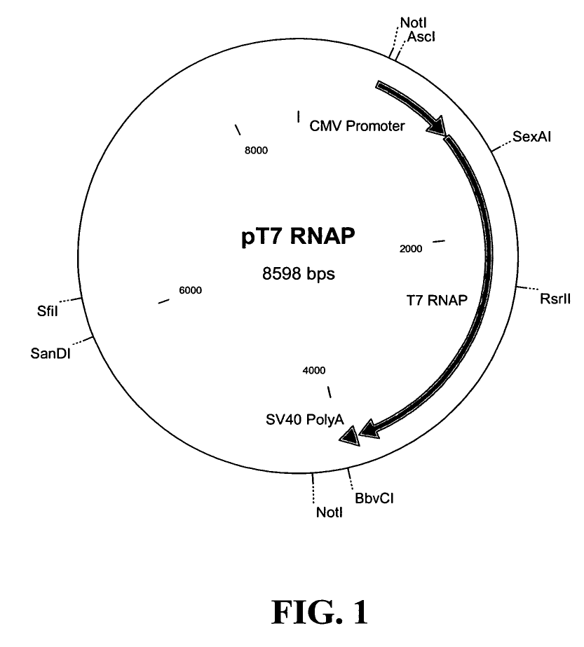

Construction of CMV-T7 RNAP vector: A unique eukaryotic expression plasmid was developed, containing the CMV promoter, a polylinker sequence for easy insertion of genes to be expressed, and a sequence directing transcriptional termination derived from the SV40 virus poly-A signal sequence (FIG. 1). This entire expression cassette is easily removed from the plasmid backbone via digestion with NotI endonuclease (FIG. 1). The E. coli strain BL21(DE3) expresses T7 RNA polymerase and provides a source of this gene (see Moffatt et al. (1984) J. Mol. Biol., 173:265-269; and U.S. Pat. No. 4,952,496 to Studier et al., “Cloning and expression of the gene for bacteriophage T7 RNA polymerase”, issued 28 Aug. 1990, which are incorporated by reference in their entirety herein). The T7 RNAP gene is cloned by polymerase chain reaction using sequence-specific oligonucleotide primers containing restriction endonuclease compatible ends, and genomic DNA from the bacterial strain as a template, and subsequently inserted into the CMV eukaryotic expression plasmid, using standard laboratory procedures (Molecular Cloning: A Laboratory Manual, Joseph Sambrook et al., Cold Spring Harbor Laboratory, 2001, 999 pp.; Short Protocols in Molecular Biology, Frederick M. Ausubel et al. (editors), John Wiley & Sons, 2002, 1548 pp.). The resulting CMV-T7 RNAP vector is transfected into eukaryotic cell cultures (such as, but not limited to, the human cell lines HeLa and 293, and the murine cell line 3T3) to demonstrate expression by Western blots or other suitable methods as known in the art.

Transgenic mouse production: A DNA construct for microinjection is prepared from the CMV-T7 RNAP plasmid as follows. The CMV-T7 RNAP expression cassette is removed and linearized via digestion with NotI endonuclease. The linearized DNA construct is purified using standard laboratory techniques (combination of glass powder and anion-exchange membrane purifications) and resuspended in buffer containing 10 millimolar Tris (pH 7.4) and 0.2 millimolar EDTA at a DNA concentration of 1 microgram per microliter. This DNA construct preparation is microinjected into the pronucleus of fertilized mouse ova (C57BL/6×5JL or inbred FVB mouse strains) as previously described (Hammer et al. (1986) J. Anim. Sci., 63:269-278; and Palmiter and Brinster (1986) Ann. Rev. Genetics, 20:465-499, which are incorporated by reference in their entirety herein). Transgenic ova are transplanted into recipient pseudopregnant female mice and allowed to develop to term to produce founder transgenic mice, which may optionally be further bred, to each other or to wild-type individuals, to obtain homozygous or hemizygous F1 transgenic vertebrates.

### Example 2

### Preparation of a Construct For Expression in a Transgenic Vertebrate

The following non-limiting example describes a system to obtain a construct for use in expressing a protein or in producing an antibody against an antigen in a transgenic vertebrate whose genome includes a bacteriophage RNA polymerase. Such constructs include the following elements operably linked: a promoter sequence cognate to the bacteriophage RNA polymerase transgene, a eukaryotic ribosome recognition site, and a sequence encoding the protein or antigen to be expressed. The construct optionally includes any combination of a stop codon, a tag sequence, and a poly-adenosine tail. The construct may be linear or circular. Circular constructs preferably include a stop codon. Linear constructs need not include a stop codon. Constructs may include a tag sequence. Constructs may also include a poly-adenosine tail.

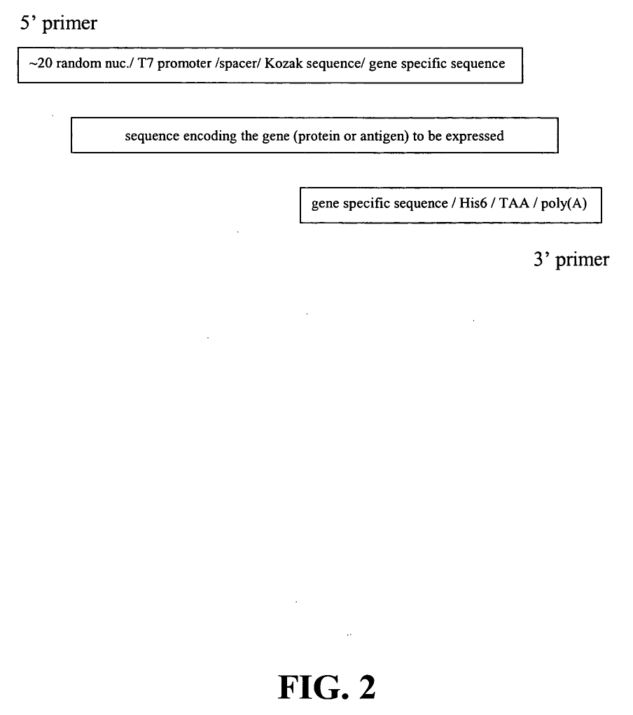

The specific system described yields a linear construct for use in a vertebrate that is transgenic for T7 RNA polymerase. This construct includes the following elements operably linked: a T7 RNA polymerase promoter (the cognate promoter sequence); a Kozak sequence (the eukaryotic ribosome recognition site); the sequence encoding the protein or antigen to be expressed; a sequence encoding a poly-histidine tag (the optional tag sequence); a stop codon; and a poly-adenosine tail (FIG. 2). For each sequence encoding the protein or antigen to be expressed, a pair of oligonucleotide PCR primers is synthesized containing sequences specific to the protein or antigen to be expressed, and sequences required for proper expression. The 5′ primer contains (starting at the 5′ end) approximately 20 non-specific nucleotides (to provide a structure for assembly of the transcriptional complex), a T7 promoter (also approximately 20 nucleotides) (see Rosa (1979) Cell, 16:815-825; and U.S. Pat. No. 4,952,496 to Studier et al., “Cloning and expression of the gene for bacteriophage T7 RNA polymerase”, issued 28 Aug. 1990, which are incorporated by reference in their entirety herein), a Kozak sequence (see “Extracting Kozak Consensus Sequence Using Kleisli”, Chen et al., available online at www.bionet.nsc.ru/bgrs/thesis/61/, accessed 28 Oct. 2003; Kozak (1982) Biochem. Soc. Symp., 47:113-128; Kozak (1982) J. Virol., 42:467-473, and Kozak (1987) Nucleic Acids Res., 15:8125-8148, which are incorporated by reference in their entirety herein) and 17-20 additional nucleotides corresponding to the beginning of the sequence encoding the protein or antigen to be expressed. The 3′ primer (starting at the 3′ end) contains 17-20 nucleotides corresponding to the end of the sequence encoding the protein or antigen to be expressed, 18 nucleotides (6 repeats of the sequence CAT) corresponding to a hexa-histidine tag (to be used for purification of the protein and confirmation of protein production via Western blot analysis), a stop codon for translation (TAA), and approximately 20 adenosines (to serve as a short poly-adenosine tail for added message stability) (FIG. 2).

Amplification of linear templates is performed using standard PCR protocols, and using a proof-reading polymerase (such as a Pfu polymerase, for example, PfuUltra™ High-Fidelity DNA Polymerase, catalogue number 600380, Stratagene, Inc., La Jolla, Calif., USA). Amplification can be carried out in single reactions, or in multiple parallel reactions, such as in 96-well or 384-well formats. After amplification, the production of correctly sized templates is confirmed via agarose gel electrophoresis.

### Example 3

### Expression and Purification of an Antibody in a Transgenic Vertebrate

The following non-limiting example describes the expression of a protein in a transgenic vertebrate whose genome comprises a bacteriophage RNA polymerase transgene. In this example, the protein is an antibody, which is produced by and isolated from the transgenic vertebrate.

Antigens: Six antigens for which well-characterized monoclonal antibodies are available are used. These include: (1) IKKα and IKKβ (cytoplasmic proteins involved in NF-κB signaling); (2) Dnmt1 and Dnmt3a (nuclear DNA methyltransferases); and (3) RANK and RANKL (cell membrane associated proteins). Highly specific antibodies against these proteins have been generated by traditional immunization of recombinant proteins. The cDNA encoding each protein is used as a sequence encoding the antigen to be expressed. Linear immunogenic constructs, each containing a sequence encoding the antigen to be expressed, are generated for DNA immunization of a vertebrate that is transgenic for T7 RNA polymerase. The efficiencies of two DNA immunization protocols are directly compared to protein antigen immunization.

DNA immunization by intramuscular injection: In one immunization protocol, three T7 transgenic mice per antigen are immunized by intramuscular injection. The transgenic mice are immunized with linear PCR products containing T7 promoter, the antigenic sequence, and a terminator sequence, all operably linked. In the T7 transgenic mouse, T7 polymerase-mediated transcription is cytoplasmic and not limited by nuclear transport of message. Additionally, the bulk of plasmid sequence is not present in the immunizing DNA. Thus, an amount of plasmid DNA smaller than the normal 50 micrograms used for intramuscular immunization is used. Approximately 1-5 micrograms of PCR product in 50 microliters phosphate buffered saline is injected into each quadriceps muscle using a disposable insulin syringe equipped with a 27-gauge needle (Manthorpe et al. (1993) Hum. Gene Ther., 4:419-431; and Wolff et al. (1990) Science, 247:1465-1468, which are incorporated in their entirety herein).

DNA immunization by gold particles: In an alternative immunization protocol, three T7 transgenic mice per antigen are immunized by DNA delivery using a gene gun. DNA-coated gold beads are prepared by combining 1-5 micrograms of PCR product and 100 microliters of 0.1 molar spermidine with 50 micrograms 1.6 micrometer gold beads. DNA is precipitated onto the beads by slowly adding 200 microliters CaCl₂ while vortexing (Eisenbraun et al. (1993) DNA Cell Biol., 12:791-797; Conry et al. (1996) Gene Ther., 3:67-74, which are incorporated in their entirety herein). Coated beads are washed, resuspended, and coated to the inner surface of Gold-Coat tubing (catalogue number 165-2431, BioRad, Hercules, Calif., USA) according to the manufacturer's recommendation. DNA-coated gold particles are propelled into the shaved thoracic and abdominal region of each T7 transgenic mouse, using the helium-driven Accell gene gun (Agracetus, Inc., Middletown, Wis.) or Helios gene gun (Biorad, Hercules, Calif., USA) on days 0, 4, 6, 8, and 11 (Kilpatrick et al. (1998) Hybridoma, 17:569-576, which is incorporated in its entirety herein).

Protein immunization: In parallel experiments; three B6SJLF2 mice are immunized with corresponding recombinant protein antigens, which are produced in bacteria as proteins tagged for purification purposes with glutathione S-transferase (GST) or a hexa-histidine tag. Each mouse is immunized intraperitoneally with 50-100 micrograms recombinant protein, pre-dialyzed in phosphate-buffered saline and mixed with an equal volume of Ribi adjuvant (Immunochem Research Inc., Hamilton, Mont., USA) to give a total immunization volume of 100 microliters. The mice are immunized on days 1, 14, and 28, and a booster immunization given three days before fusion. The mice are bled on day 21 and the sera tested for reactivity against the recombinant proteins by ELISA. The recombinant protein antigens are also used for testing the serum by Western blotting. This allows comparison of the efficiency of DNA immunization versus traditional protein antigen immunization protocol.

### Example 4

### Screening for Antibody Titer

Serum antibody assay: The antibody response is measured by titering sera from immunized mice on 96 well, ½ area microtiter plates (Corning/Costar, Inc., Corning, N.Y.) coated with recombinant protein antigens (produced in bacteria or expressed in vitro using transcription-coupled translation (TnT) system (catalogue number L1170, Promega, Madison, Wis., USA) at 2.0 microgram per milliliter in 50 microliters of borate-buffered saline (BBS) (89 millimolar boric acid, 90 millimolar NaCl, pH 8.3) per well. After overnight incubation at 4 degrees Celsius, plates are washed with BBST (1×BBS with 0.1% Tween 20), blocked for 1 hour with 100 microliters of 5% non-fat milk in BBS. Two-fold serial dilutions of sera in 5% milk, starting at 1:20, are added to each well at 50 microliters per well. Plates are incubated at room temperature for 4 hours, and washed with BBST. An alkaline phosphatase-cojugated goat anti-mouse IgG-Fc (catalogue number 115-055-062, Jackson Immunoresearch, West Grove, Pa., USA) diluted 1:1000 in 5% non-fat milk is added at 50 microliters per well. Plates are incubated for 1 hour at room temperature, washed, and 50 microliters per well of 4-nitrophenyl phosphate (pNPP) substrate is added. The absorption at 405 nanometers is read after incubation for 30 minutes at room temperature. An optical density between about 1 to about 2 (at 1:1000 dilution) at 405 nanometers is generally considered to indicate a good titer for antibody in the sera. Antigen-specific hybridomas from fusion done with splenocytes yield a serum antibody titer giving an optical density at 405 nanometers of more than about 1.

Analysis on arrays: A high-throughput screening system using multiple antigens on membranes is used to test the generation of immune responses against the antigens. Aliquots of purified protein antigens corresponding to fifty nanograms are transferred to nylon membranes using multi-channel micropipettes and a membrane blotting/drying apparatus to ensure uniformity of the individual samples within the array. Additionally, similar aliquots are transferred to multi-well plastic plates and allowed to completely coat each well. To demonstrate that the applied protein antigen samples will act as effective antigenic targets in a screening system, dot-blot western analysis of this membrane array and a simple ELISA assay on the plates are performed, using a set of antibodies (preferably monoclonal, or a combination of monoclonal and polyclonal) corresponding to these protein antigens. These antibodies are used individually and in sets, in order to demonstrate specificity and non-interference of each interaction.

### Example 5

### Monoclonal Antibody Production Using Transgenic Vertebrates

Fusion and hybridoma selection: Parallel hybridoma fusions are carried out. Mice with high antibody titer for a specific antigen (one non-transgenic mouse immunized with recombinant protein and one T7 RNAP transgenic mouse immunized by the corresponding PCR DNA immunogenic construct) are sacrificed and spleen cells (splenocytes) are separately fused to myeloma cells to generate hybridomas secreting monoclonal antibodies. FO-SF-II non-secretory myeloma cells (de St. Groth (1980) Transplant Proc., 12:447-450), maintained in log phase, are mixed with the single cell suspension of immunized mouse splenocytes. The splenocyte/myeloma cell mixture is fused by following a standard polyethylene glycol (PEG)-mediated fusion protocol (Kohler and Milstein (1992) Biotechnology, 24:524-526; and Kohler et al. (1976) Eur. J. Immunol., 6:292-295, which are incorporated by reference in their entirety herein). The splenocytes are mixed with the FO-SF-II myeloma cells at a ratio of 10:1. The cell mixture is washed at least three times in serum-free Iscove's Modified Dulbecco's Medium (IMDM). The cells are pelleted and 50% polyethylene glycol (PEG) added drop-wise to the cell pellet with constant but gentle agitation. After careful addition of IMDM medium supplemented with Fetal Calf Serum, the cells are incubated at 37 degrees Celsius for about 4 to about 5 hours. The cells are washed once to remove the PEG, mixed with a feeder layer of cells obtained from a control mouse spleen, and plated in 96-well plates.

The following day, the fused cells are placed in medium containing hypoxanthine aminopterin thymidine (HAT) to select for hybridomas. The medium is changed twice a week until hybridoma colonies are large enough for screening. Hybridoma wells that are positive by ELISA (as described below under the heading “Screening hybridoma supernatants by ELISA”) against the antigen are expanded to 24-well plates in preparation for cloning by limiting dilution. When the wells become confluent, the supernatant from each well is screened by Western blotting (as described below under the heading “Western blot analysis”) to test the specificity of the antibody. If the hybridoma well is positive by Western blotting, the cells are cloned by limiting dilution at one cell per well. Supernatants from confluent wells are screened again by ELISA and Western blotting. Only positive wells are expanded and cloned once again. The HAT selection pressure is maintained throughout the cloning procedure until desirable hybridoma clones are selected.

Screening hybridoma supernatants by ELISA: For each antigen (whether immunization was by recombinant protein antigen or the corresponding DNA immunogenic construct), hybridoma supernatants are initially screened by enzyme-linked immunosorbent assay (ELISA). Ninetysix-well microtiter plates are coated with 50 nanograms per well of specific antigen diluted in 100 microliters phosphate-buffered saline (PBS), incubated overnight at 4 degrees Celsius, and washed to remove excess antigen. Non-specific binding of antibody is blocked by incubating with 1% bovine serum albumin in PBS. Each hybridoma supernatant to be tested for antibody production is added to a separate well of the microtiter plate and incubated for about 1 hour. The wells are washed and incubated with alkaline phosphatase-conjugated goat anti-mouse IgG (catalogue number 115-055-062, Jackson Immunoresearch, West Grove, Pa., USA) for about 1 hour. After washing off unbound secondary antibody, the wells are incubated with a solution of 4-nitrophenyl phosphate (pNPP) substrate. Chromogenic reaction of the positive clones is estimated by measuring the optical density at 405 nanometers using an ELISA plate reader. Hybridoma supernatants that react only with the appropriate antigen are further processed. Antibodies from all the selected hybridoma clones are further characterized by Western blot analysis (as described below under the heading “Western blot analysis”) and by immunoprecipitation, using as antigen cell lysates from cell lines that express the native protein (for example, for IKKα and IKKβ, HeLa cell lysate is used).

Western blot analysis: Monoclonal antibodies that react only with the appropriate antigen by ELISA are tested for specific reactivity against the recombinant protein antigen as well as native protein from cell lysates by western blotting. Cell pellets from the various cell lines are solubilized by incubating in lysis buffer containing 0.1% Triton X-100, supplemented with a cocktail of protease inhibitors (benzamidine hydrochloride, aprotinin, leupeptin, pepstatin A, and phenylmethanesulfonyl fluoride). After incubating on ice for 30 minutes, the insoluble fraction is pelleted by centrifugation at 15,000 rpm at 4 degrees Celsius for 20 minutes and the supernatant (soluble cell lysate) retained for western blot analysis. Protein concentrations are estimated by the Lowry method (Lowry et al. (1951) J. Biol. Chem., 193:265-275). For each cell line, equal amount of total protein (200 milligrams) are loaded per 1-well mini SDS-PAGE preparative gel (Bio-Rad Laboratories, Cambridge, Mass., USA) and resolved. The resolved proteins are electroblotted onto Immobilon P membranes (Millipore Corporation, Bedford, Mass., USA) at between about 0.5 to about 0.75 amperes for 1 hour. After transfer, the blots are stained with 0.05% amido black in 10% methanol solution, and the position of molecular weight standards is marked. Multiple vertical strips of the blots are cut and each strip used to test a single primary antibody (either a purified monoclonal antibody at a standard concentration of 2 micrograms per milliliter or a hybridoma tissue culture supernatant diluted 1:3 with 5% nonfat dry milk in Tris-buffered saline containing 0.1% Tween-20 (TBST)). The strips are initially blocked for at least 1 hour with 5% non-fat dry milk in TBST, then incubated at 4 degrees Celsius overnight with primary antibody in 1% non-fat milk in TBST. The strips are washed several times in TBST, and incubated with horseradish peroxidase-conjugated anti-mouse IgG secondary antibody (catalogue number 554002, PharMingen, Calif.) for 60 minutes at room temperature. After several extensive washes, antibody immunoreactivity is visualized using the Luminol SuperSignalR chemiluminescence kit, (catalogue number 34080, Pierce Chemical Company, Rockford, Ill., USA), and the strips are exposed to autoradiographic film (catalogue number 8941114, Eastman Kodak Company, Rochester, N.Y., USA) and developed using a Konica SRX-101A film developer (Konica, San Diego, Calif., USA).

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

What is claimed is:

1. A transgenic vertebrate whose genome comprises a bacteriophage RNA polymerase as a transgene, wherein said bacteriophage RNA polymerase transgene is capable of being expressed in at least one cell of said transgenic vertebrate.

2. The transgenic vertebrate of claim 1, wherein said vertebrate is a bird.

3. The transgenic vertebrate of claim 1, wherein said vertebrate is a fish.

4. The transgenic vertebrate of claim 1, wherein said vertebrate is an amphibian.

5. The transgenic vertebrate of claim 1, wherein said vertebrate is a mammal.

6. The transgenic vertebrate of claim 5, wherein said mammal is selected from the group consisting of non-human primates, dogs, cats, sheep, pigs, goats, cattle, horses, ferrets, rats, rabbits, hamsters, gerbils, and mice.

7. The transgenic vertebrate of claim 5, wherein said mammal is a mouse.

8. The transgenic vertebrate of claim 1, wherein said bacteriophage RNA polymerase is selected from the group consisting of T7, SP6, and T3 RNA polymerases.

9. The transgenic vertebrate of claim 1, wherein said bacteriophage RNA polymerase is a T7 RNA polymerase.

10. The transgenic vertebrate of claim 1, wherein said transgene is operably linked to a promoter.

11. The transgenic vertebrate of claim 10, wherein said promoter is a constitutive promoter.

12. The transgenic vertebrate of claim 10, wherein said promoter is an inducible promoter.

13. A method of expressing a protein in a transgenic vertebrate whose genome comprises a bacteriophage RNA polymerase transgene, comprising:
a) providing a construct comprising the following elements operably linked:
i) a promoter sequence cognate to said bacteriophage RNA polymerase,
ii) a eukaryotic ribosome recognition sequence, and
iii) a sequence encoding said protein;
b) introducing said construct into at least one cell of said transgenic vertebrate; and
c) providing conditions whereby said transgenic vertebrate expresses said protein.

14. The method of claim 13, wherein said transgenic vertebrate is a bird.

15. The method of claim 13, wherein said transgenic vertebrate is a fish.

16. The method of claim 13, wherein said vertebrate is an amphibian.

17. The method of claim 13, wherein said transgenic vertebrate is a mammal.

18. The method of claim 17, wherein said mammal is selected from the group consisting of non-human primates, dogs, cats, sheep, pigs, goats, cattle, horses, ferrets, rats, rabbits, hamsters, gerbils, and mice.

19. The method of claim 17, wherein said mammal is a mouse.

20. The method of claim 13, wherein said bacteriophage RNA polymerase is selected from the group consisting of T7, SP6, and T3 RNA polymerases.

21. The method of claim 13, wherein said bacteriophage RNA polymerase is a T7 RNA polymerase.

22. The method of claim 13, wherein said transgene is operably linked to a promoter.

23. The method of claim 22, wherein said promoter is a constitutive promoter.

24. The method of claim 22, wherein said promoter is an inducible promoter.

25. The method of claim 13, wherein said construct further comprises a stop codon.

26. The method of claim 13, wherein said construct further comprises a tag sequence.

27. The method of claim 13, wherein said construct further comprises a poly-adenosine tail.

28. The method of claim 13, further comprising the step of isolating said protein.

29. A protein produced by the method of claim 28.

30. A method to produce at least one antibody against an antigen in a transgenic vertebrate whose genome comprises a bacteriophage RNA polymerase transgene, comprising:
a) providing a immunogenic construct comprising the following elements operably linked:
i) a promoter sequence cognate to said bacteriophage RNA polymerase,
ii) a eukaryotic ribosome recognition sequence, and
iii) a sequence encoding said antigen;
b) introducing said immunogenic construct into at least one cell of said transgenic vertebrate; and
c) providing conditions whereby said transgenic vertebrate produces said at least one antibody against said antigen.

31. The method of claim 30, wherein said transgenic vertebrate is a bird.

32. The method of claim 30, wherein said transgenic vertebrate is a fish.

33. The method of claim 30, wherein said transgenic vertebrate is an amphibian.

34. The method of claim 30, wherein said transgenic vertebrate is a mammal.

35. The method of claim 34, wherein said mammal is selected from the group consisting of non-human primates, dogs, cats, sheep, pigs, goats, cattle, horses, ferrets, rats, rabbits, hamsters, gerbils, and mice.

36. The method of claim 34, wherein said mammal is a mouse.

37. The method of claim 30, wherein said bacteriophage RNA polymerase is selected from the group consisting of T7, SP6, and T3 RNA polymerases.

38. The method of claim 30, wherein said bacteriophage RNA polymerase is a T7 RNA polymerase.

39. The method of claim 30, wherein said transgene is operably linked to a promoter.

40. The method of claim 39, wherein said promoter is a constitutive promoter.

41. The method of claim 39, wherein said promoter is an inducible promoter.

42. The method of claim 30, wherein said immunogenic construct further comprises a stop codon.

43. The method of claim 30, wherein said immunogenic construct further comprises a poly-adenosine tail.

44. The method of claim 30, further comprising the step of isolating said at least one antibody as a polyclonal antibody.

45. An antibody produced by the method of claim 44.

46. The method of claim 30, further comprising the steps of collecting spleen cells from said transgenic vertebrate, making at least one hybridoma from said spleen cells, and isolating said at least one antibody as a monoclonal antibody from said at least one hybridoma.

47. An antibody produced by the method of claim 46.

48. The method of claim 30, further comprising the steps of collecting at least one egg from said bird and isolating said at least one antibody as an IgY antibody from yolk of said at least one egg.

49. An antibody produced by the method of claim 48.

50. A method to produce a transgenic vertebrate whose genome comprises a bacteriophage RNA polymerase transgene, comprising:
a) introducing into the pronucleus of a fertilized ovum of a vertebrate a construct comprising a bacteriophage RNA polymerase as a transgene;
b) transplanting said ovum into a female of said vertebrate; and
c) allowing said ovum to develop to term, thereby producing a founder transgenic vertebrate individual.

51. The method of claim 50, wherein said transgenic vertebrate is a bird.

52. The method of claim 50, wherein said transgenic vertebrate is a fish.

53. The method of claim 50, wherein said transgenic vertebrate is an amphibian.

54. The method of claim 50, wherein said transgenic vertebrate is a mammal.

55. The method of claim 55, wherein said mammal is selected from the group consisting of non-human primates, dogs, cats, sheep, pigs, goats, cattle, horses, ferrets, rats, rabbits, hamsters, gerbils, and mice.

56. The method of claim 55, wherein said mammal is a mouse.

57. The method of claim 50, wherein said bacteriophage RNA polymerase is selected from the group consisting of T7, SP6, and T3 RNA polymerases.

58. The method of claim 50, wherein said bacteriophage RNA polymerase is a T7 RNA polymerase.

59. The method of claim 50, wherein said transgene is operably linked to a promoter.

60. The method of claim 59, wherein said promoter is a constitutive promoter.

61. The method of claim 59, wherein said promoter is an inducible promoter.

62. The method of claim 50, further comprising the step of breeding said founder transgenic vertebrate individual to obtain F1 transgenic vertebrates homozygous or hemizygous for said transgene.

63. A method to produce a transgenic vertebrate whose genome comprises a bacteriophage RNA polymerase transgene, comprising:
a) providing a transgene construct comprising a bacteriophage RNA polymerase as a transgene
b) introducing said transgene construct into embryonic stem cells of said vertebrate;
c) selecting at least one embryonic stem cell that has incorporated said transgene by recombination;
d) introducing said at least one embryonic stem cell that has incorporated said transgene by recombination into at least one blastocyst of said vertebrate;
e) transplanting said at least one blastocyst into a pseudopregnant female of said vertebrate; and
f) allowing said at least one blastocyst to develop to term, thereby producing at least one chimeric founder transgenic vertebrate individual.

64. The method of claim 63, wherein said transgenic vertebrate is a bird.

65. The method of claim 63, wherein said transgenic vertebrate is a fish.

66. The method of claim 63, wherein said transgenic vertebrate is an amphibian.

67. The method of claim 63, wherein said transgenic vertebrate is a mammal.

68. The method of claim 67, wherein said mammal is selected from the group consisting of non-human primates, dogs, cats, sheep, pigs, goats, cattle, horses, ferrets, rats, rabbits, hamsters, gerbils, and mice.

69. The method of claim 67, wherein said mammal is a mouse.

70. The method of claim 63, wherein said bacteriophage RNA polymerase is selected from the group consisting of T7, SP6, and T3 RNA polymerases.

71. The method of claim 63, wherein said bacteriophage RNA polymerase is a T7 RNA polymerase.

72. The method of claim 63, wherein said transgene is operably linked to a promoter.

73. The method of claim 72, wherein said promoter is a constitutive promoter.

74. The method of claim 72, wherein said promoter is an inducible promoter.

75. The method of claim 63, wherein said recombination is homologous.

76. The method of claim 63, wherein said recombination is heterologous.

77. The method of claim 63, further comprising the step of breeding said chimeric founder transgenic vertebrate individuals to obtain F1 transgenic vertebrates homozygous or hemizygous for said transgene.

78. The method of claim 63, wherein said transgene construct further comprises a viral vector.

79. The method of claim 63, wherein said viral vector is selected from the group consisting of a lentivirus, a retrovirus, an adenovirus, an adeno-associated virus, a replication-deficient virus, and a hybrid viral vector.

80. A method to produce a transgenic vertebrate whose genome comprises a bacteriophage RNA polymerase transgene, comprising:
a) providing a transgene construct comprising a bacteriophage RNA polymerase as a transgene
b) introducing said transgene construct into at least one embryonic cell of said vertebrate;
c) selecting at least one embryonic cell that has incorporated said transgene by recombination;
d) allowing said at least one embryonic cell that has incorporated said transgene by recombination to develop into at least one blastocyst of said vertebrate;
e) transplanting said at least one blastocyst into a pseudopregnant female of said vertebrate; and
f) allowing said at least one blastocyst to develop to term, thereby producing at least one chimeric founder transgenic vertebrate individual.

81. The method of claim 80, wherein said transgenic vertebrate is a bird.

82. The method of claim 80, wherein said transgenic vertebrate is a fish.

83. The method of claim 80, wherein said transgenic vertebrate is an amphibian.

84. The method of claim 80, wherein said transgenic vertebrate is a mammal.

85. The method of claim 84, wherein said mammal is selected from the group consisting of non-human primates, dogs, cats, sheep, pigs, goats, cattle, horses, ferrets, rats, rabbits, hamsters, gerbils, and mice.

86. The method of claim 84, wherein said mammal is a mouse.

87. The method of claim 80, wherein said bacteriophage RNA polymerase is selected from the group consisting of T7, SP6, and T3 RNA polymerases.

88. The method of claim 80, wherein said bacteriophage RNA polymerase is a T7 RNA polymerase.

89. The method of claim 80, wherein said transgene is operably linked to a promoter.

90. The method of claim 89, wherein said promoter is a constitutive promoter.

91. The method of claim 89, wherein said promoter is an inducible promoter.

92. The method of claim 80, wherein said recombination is homologous.

93. The method of claim 80, wherein said recombination is heterologous.

94. The method of claim 80, further comprising the step of breeding said chimeric founder transgenic vertebrate individuals to obtain F1 transgenic vertebrates homozygous or hemizygous for said transgene.

95. The method of claim 80, wherein said transgene construct further comprises a viral vector.

96. The method of claim 95, wherein said viral vector is selected from the group consisting of a lentivirus, a retrovirus, an adenovirus, an adeno-associated virus, a replication-deficient virus, and a hybrid viral vector.

97. The method of claim 80, wherein said at least one embryonic cell is at least one morula cell.

98. A method to produce a transgenic vertebrate whose genome comprises a bacteriophage RNA polymerase transgene, comprising:
a) providing a transgene construct comprising a bacteriophage RNA polymerase as a transgene
b) introducing said transgene construct into at least one male germ-line stem cell of said vertebrate;
c) selecting said at least one male germ-line stem cell that has incorporated said transgene by recombination;
d) introducing said at least one male germ-line stem cell that has incorporated said transgene by recombination into a recipient male of said vertebrate;
e) allowing said at least one male germ-line stem cell that has incorporated said transgene by recombination to develop to maturity in said recipient male, thereby producing at least one mature transgenic spermatozoon; and
f) breeding said recipient male carrying said at least one mature transgenic spermatozoon to obtain F1 transgenic vertebrates hemizygous for said transgene.

99. The method of claim 98, wherein said transgenic vertebrate is a bird.

100. The method of claim 98, wherein said transgenic vertebrate is a fish.

101. The method of claim 98, wherein said transgenic vertebrate is an amphibian.

102. The method of claim 98, wherein said transgenic vertebrate is a mammal.

103. The method of claim 102, wherein said mammal is selected from the group consisting of non-human primates, dogs, cats, sheep, pigs, goats, cattle, horses, ferrets, rats, rabbits, hamsters, gerbils, and mice.

104. The method of claim 102, wherein said mammal is a mouse.

105. The method of claim 98, wherein said bacteriophage RNA polymerase is selected from the group consisting of T7, SP6, and T3 RNA polymerases.

106. The method of claim 98, wherein said bacteriophage RNA polymerase is a T7 RNA polymerase.

107. The method of claim 98, wherein said transgene is operably linked to a promoter.

108. The method of claim 107, wherein said promoter is a constitutive promoter.

109. The method of claim 107, wherein said promoter is an inducible promoter.

110. The method of claim 98, wherein said recombination is homologous.

111. The method of claim 98, wherein said recombination is heterologous.

112. The method of claim 98, wherein said transgene construct further comprises a viral vector.

113. The method of claim 112, wherein said viral vector is selected from the group consisting of a lentivirus, a retrovirus, an adenovirus, an adeno-associated virus, a replication-deficient virus, and a hybrid viral vector.
